(19) United States
(12) Patent Application Publication
(10) Pub. No.: US 2013/0217760 A1
(43) Pub. Date: Aug. 22, 2013

(54) METHOD FOR ENHANCING THERAPEUTIC EFFECT OF ANTICANCER THERAPIES

(71) Applicant: BOTAMEDI INC., (US)

(72) Inventors: Hyeon-Cheol SHIN, Daejeon (KR); Hye-Jeong HWANG, Daejeon (KR); Seong-Ho KIM, Daejeon (KR)

(73) Assignee: BOTAMEDI INC., Jeju-city (KR)

(21) Appl. No.: 13/768,333

(22) Filed: Feb. 15, 2013

(30) Foreign Application Priority Data
Feb. 17, 2012 (KR) 1020120016155

(51) Int. Cl. A61K 31/357

(52) U.S. Cl. CPC A61K 31/357
USPC 514/452

(57) ABSTRACT

Disclosed is a method for enhancing therapeutic effect of anticancer therapies for an animal, comprising administering one or more dibenzo-p-dioxin derivative in an effective amount to the animal in anticancer therapies. The method augments therapeutic effects of an anticancer therapies, with the suppression of side effects of the anticancer therapies.

### CROSS REFERENCE TO RELATED APPLICATION(S)

This application claims the benefit of Korean Patent Application No. 10-2012-0016155, filed Feb. 17, 2012, which is hereby incorporated by reference in its entirety into this application.

### BACKGROUND OF THE INVENTION

1. Technical Field

The method relates to a method for improving an anticancer therapy. More particularly, the present invention relates to the use of a dibenzo-p-dioxin derivative in augmenting effects of an anticancer therapy, with the concomitant suppression of side effects of the anticancer therapy.

2. Description of the Related Art

With the westernization of living environment and dietary habits and the increase of the elderly population, the onset of various cancers has become increasingly prevalent. In spite of extensive research and investment, no remarkable achievements have taken place regarding the prophylaxis and therapy of cancer. Serious side effects prevent conventional anticancer agents from being effectively applied. On the whole, surgical procedures and radiotherapy are used only when cancer is in an early stage or in a local state. In addition, anticancer therapy, if applied repetitively, may promote resistance thereto, causing the cancer to be recurrent or malignant. Further, there are no effective means for regulating post-therapy metastasis or recurrence of cancer, which is closely correlated with low cancer survival rates. Moreover, chemotherapy is accompanied by significant side effects. Inter alia, anemia, digestive dysfunction, chronic fatigue, hair loss, infection, emesis, and cognitive decline are primarily induced. More serious is its production of permanent damage to major organs such as the heart, the kidneys, the bladder, the lungs, and the nerve system. This is attributed to the fact that anticancer agents attack normal cells that are in active cell division, such as blood cells, digestive cells, immune cells, gametes, dermal papilla cells, as well as cancer cells. Therefore, there is a pressing need for an agent that can, when used in combination with an anticancer therapy, augment effects of the anticancer therapy with the concomitant reduction of side effects and drug resistance.

Korean Patent Application Nos. 2006-0009315 and 2005-0040864 discloses compositions comprising dibenzo-p-dioxin derivatives for inhibiting angiotensin converting enzyme or activator protein-1 (AP-1), but nowhere is the use of dibenzo-p-dioxin derivatives for augmenting effects of anticancer therapy mentioned in the patent applications.

Leading to the present invention, intensive and thorough research into anticancer therapy resulted in the finding that dibenzo-p-dioxin derivatives, when used in combination with an anticancer therapy, augment the apoptosis of cancer cells with the concomitant reduction of side effects of the anticancer therapy.

### PRIOR ART DOCUMENT

### Patent Document

KR 10-2006-0009315
KR 10-2005-0040864

### SUMMARY OF THE INVENTION

It is therefore an object of the present invention to provide a method for enhancing therapeutic effect of anticancer therapies for an animal, with the maximization of the therapeutic effects of the anticancer therapies.

In order to accomplish the object, the present invention provides a method for enhancing therapeutic effect of anticancer therapies for an animal, comprising administering one or more dibenzo-p-dioxin derivative in an effective amount to the animal in anticancer therapies, wherein said dibenzo-p-dioxin derivative is one or more selected from the group consisting of compounds represented by the following Chemical Formulas 1 to 10

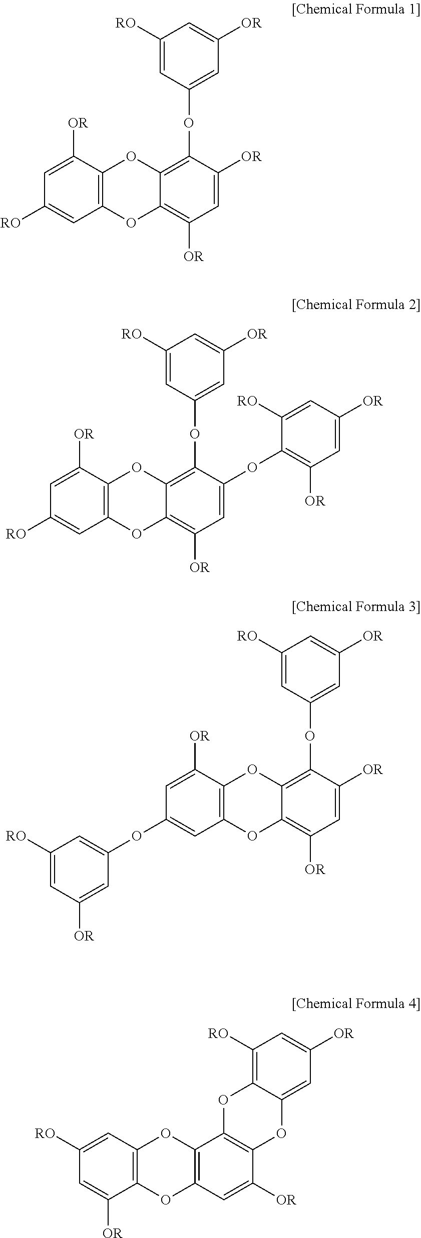

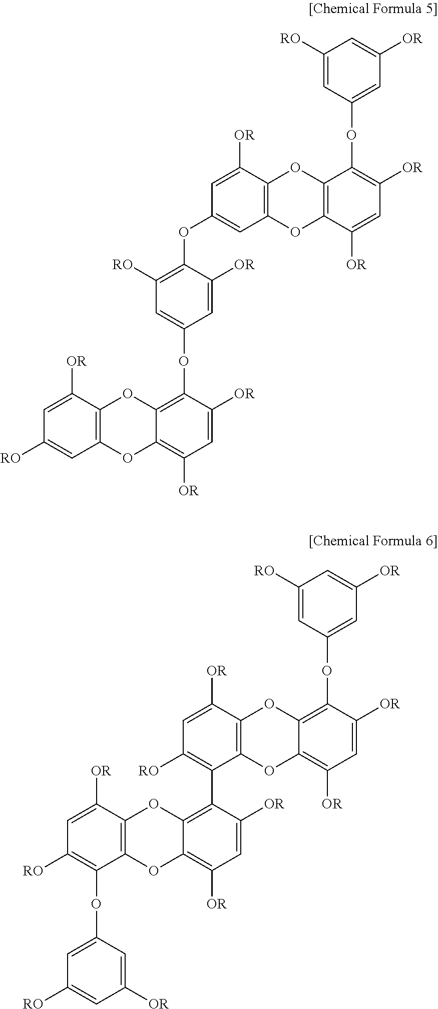

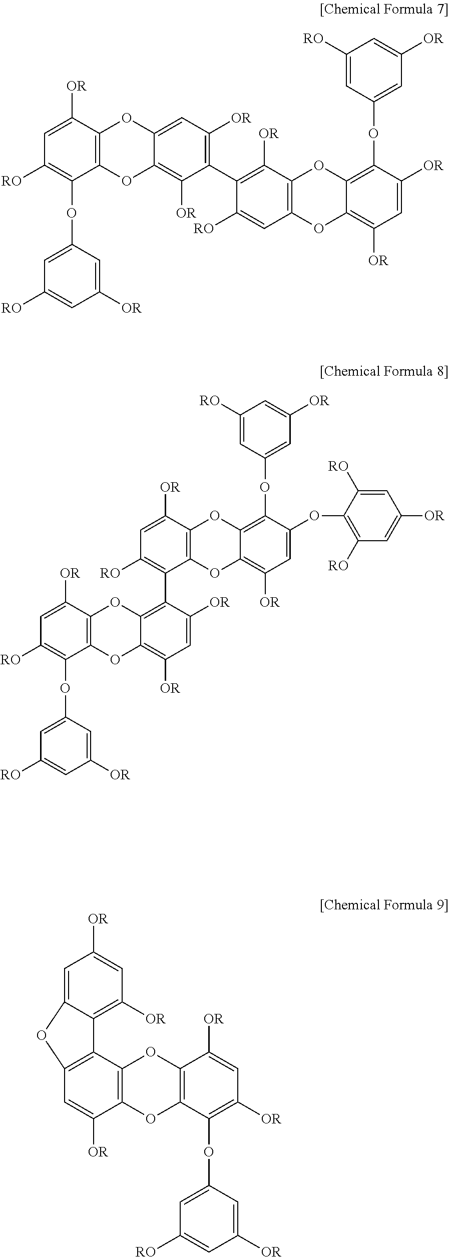

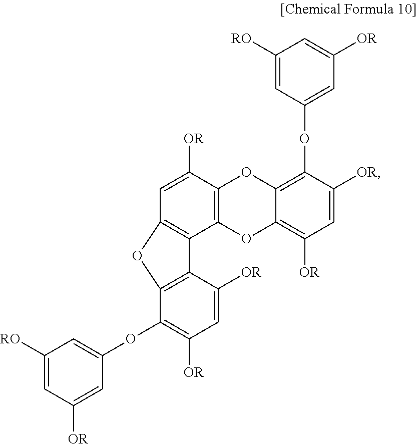

wherein Rs are independently hydrogen, alkyl of C1˜C5, alkenyl of C2˜C5, phenyl, phenylalkyl of C7˜C12, alkanoyl of C2˜C20, alkenoyl of C3˜C20, hydroxyphenyl, dihydroxyphenyl or trihydroxyphenyl.

In one embodiment of the present invention, Rs are hydrogen.

In another embodiment of the present invention, the dibenzo-p-dioxin derivative may comprise compounds represented by Chemical Formulas 3, 5, 6, and 8; compounds represented by Chemical Formulas 5, 6, 7, and 8; compounds represented by Chemical Formulas 3, 5, 6, 8, and 10; compounds represented by Chemical Formulas 1, 2, 3, 4, 5, 6, 7, 8, 9, and 10; or compounds represented by Chemical Formulas 1, 2, 3, and 10.

In a further embodiment of the present invention, the method may be applied to the anticancer therapies for breast cancer, ovarian cancer, lung cancer, liver cancer, stomach cancer, prostate cancer, thyroid cancer, colorectal cancer, or uterine cervical cancer.

In still another embodiment of the present invention, the anticancer therapies may be chemotherapies with an anticancer agent selected from the group consisting of doxorubicin, cisplatin, paclitaxel, and a combination thereof.

In a still further embodiment of the present invention, the anticancer therapy may be a radiotherapy.

In yet another embodiment of the present invention, the method enhances therapeutic effect of anticancer therapies by augmenting an apoptotic effect of the anticancer therapies on cancer cells.

In a yet further embodiment of the present invention, the method enhances therapeutic effect of anticancer therapies by reducing and/or alleviating the occurrence of cardiomyopathy caused by the anticancer therapy.

In yet still another embodiment of the present invention, the method enhances therapeutic effect of anticancer therapies by alleviating the anorexia caused by the anticancer therapy.

In a yet still further embodiment of the present invention, the enhances therapeutic effect of anticancer therapies by protecting the kidney or liver from nephrotoxicity or hepatotoxicity induced by the anticancer therapy.

The method of the present invention, when applied in combination with anticancer therapies, enhances the anticancer therapies, with the maximization of therapeutic effects of the anticancer therapies and the reduction of side effects evoked by the anticancer therapies.

### DESCRIPTION OF THE PREFERRED EMBODIMENTS

The present invention is directed towards a method for enhancing therapeutic effect of anticancer therapies by eliciting maximal therapeutic effects of the anticancer therapy.

With the aim of overcoming problems with conventional anticancer therapies, the present inventors have researched into a means that is selective for cancer cells, absolutely safe, and potently protective of normal cells. Also, the research of the present inventors has targeted the development of a composition or a method which can remarkably alleviate the toxicity and side effects of conventional anticancer therapies, augment therapeutic effects of anticancer therapies by sensitization, and prevent the recurrence or metastasis after treatment.

To achieve these goals, the present inventors conducted an intensive and thorough search for a natural component free of irritation/toxicity which has excellent antioxidant and anti-inflammatory activity and which can reduce side effects provoked by anticancer therapies, and this research resulted in finding that dibenzo-p-dioxin derivatives extracted from plants meet the requirements, which leads to the present invention.

The present invention induces the selective apoptosis of cancer cells, with the minimization of damage on normal cells, thus allowing for the effective performance of anticancer therapy. According to the present invention, anticancer therapies can be performed with the reduction of side effects by 70-90% and the augmentation of therapeutic effects by 300% or higher.

Particularly, the present invention concerns the augmentation of therapeutic effects of anticancer therapy. In accordance with an aspect thereof, the present invention addresses a composition for improving an anticancer therapy, comprising a dibenzo-p-dioxin derivative as an active ingredient. Also, the present invention envisages a method for enhancing therapeutic effect of anticancer therapies. That is, contemplated in accordance with another aspect of the present invention is a method for enhancing therapeutic effect of anticancer therapies, comprising administering a dibenzo-p-dioxin derivative in an effective amount, to be administered to an animal in need thereof. The dibenzo-p-dioxin derivative may be selected from the group consisting of compounds represented by the following Chemical Formulas 1 to 10:

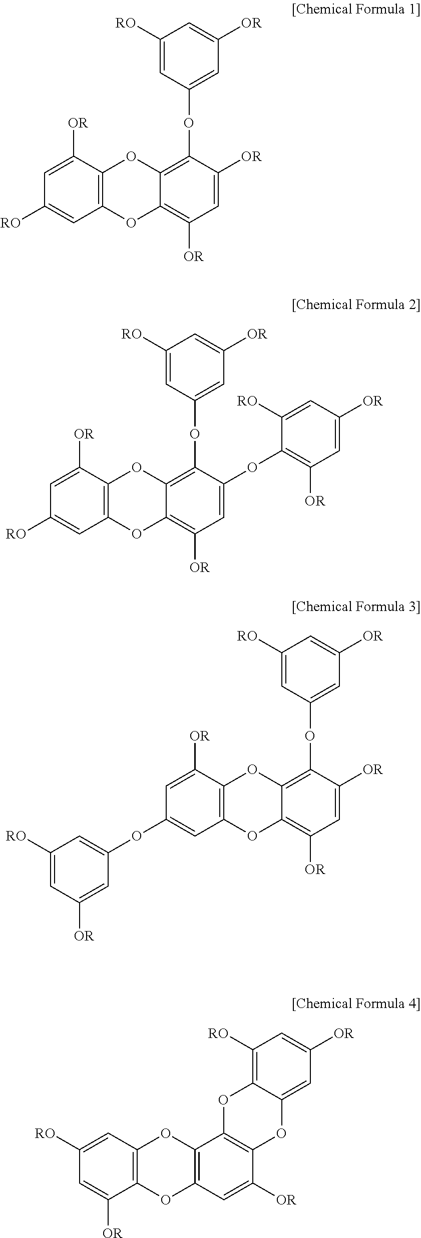

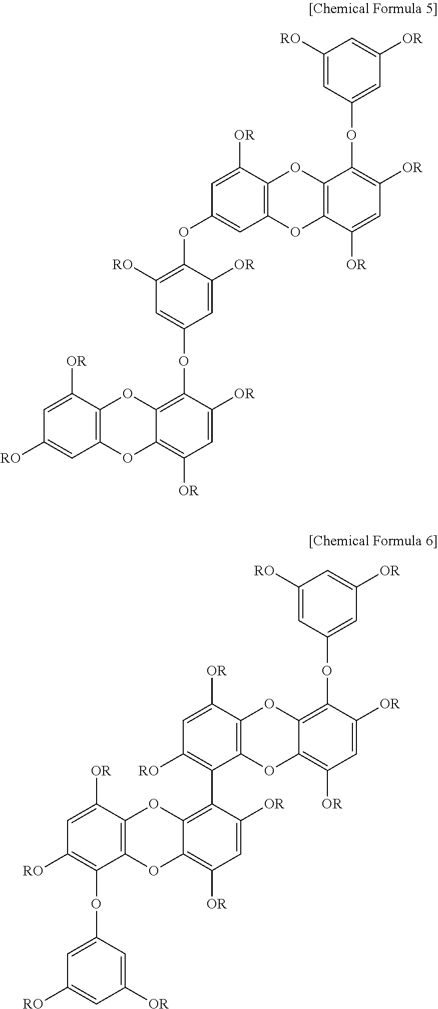

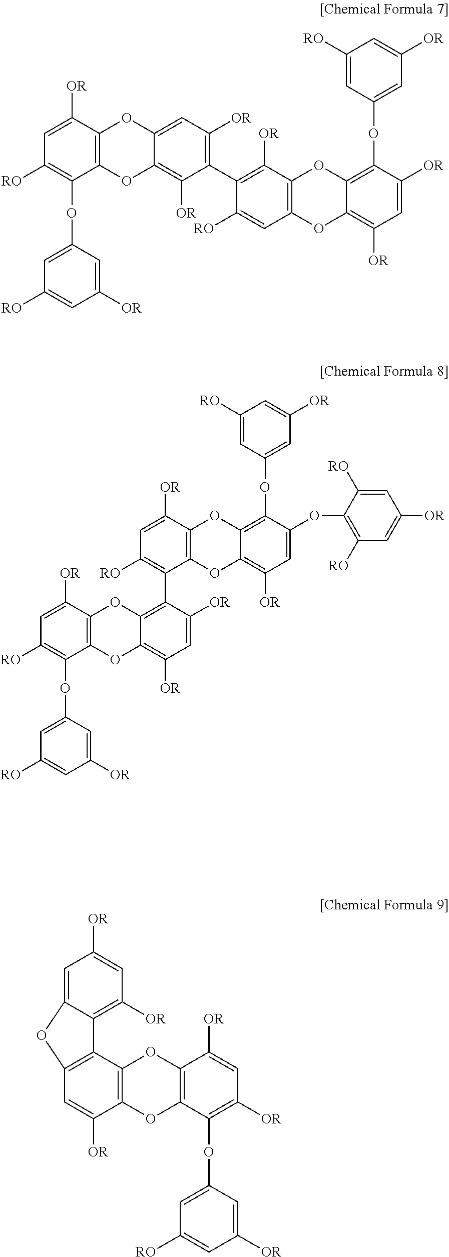

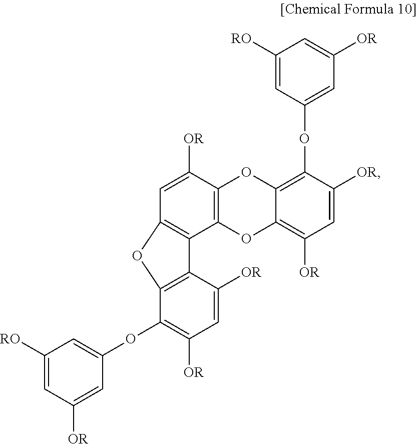

and a combination thereof.

wherein, Rs are independently hydrogen, alkyl of C1˜C5, alkenyl of C2˜C5, phenyl, phenylalkyl of C7˜C12, alkanoyl of C2˜C20, alkenoyl of C3˜C20, hydroxyphenyl, dihydroxyphenyl or trihydroxyphenyl.

In a preferred embodiment, Rs are independently hydrogen; methyl; ethenyl; benzyl; acetyl or oleoyl; 4-hydroxyphenyl; 2,4-hydroxyphenyl; or 2,4,6-trihydroxyphenyl. More preferably, Rs are hydrogen.

In another preferred embodiment, the dibenzo-p-dioxin derivative of the present invention may comprise two or more compounds selected from the group consisting of compounds represented by Chemical Formulas 3, 5, 6 and 8, with the proviso that Rs are hydrogen. In this regard, the dibenzo-p-dioxin derivative may comprise the compound of Chemical Formula 3 in an amount of 10˜60 wt. %, the compound of Chemical Formula 5 in an amount of 15˜60 wt. %, the compound of Chemical Formula 6 in an amount of 10˜40 wt. %, and the compound of Chemical Formula 8 in an amount of 5˜30 wt. %.

In a further preferred embodiment, the dibenzo-p-dioxin derivative of the present invention may comprise two or more compounds selected from the group consisting of compounds represented by Chemical Formulas 5, 6, 7 and 8, with the proviso that Rs are hydrogen. In this regard, the dibenzo-p-dioxin derivative may comprise the compound of Chemical Formula 5 in an amount of 10˜60 wt. %, the compound of Chemical Formula 6 in an amount of 15˜60 wt. %, the compound of Chemical Formula 7 in an amount of 10˜40 wt. %, and the compound of Chemical Formula 8 in 10˜40 wt. %.

In a still further preferred embodiment, the dibenzo-p-dioxin derivative of the present invention may comprise two or more compounds selected from the group consisting of compounds represented by Chemical Formulas 3, 5, 6, 8 and 10, with the proviso that Rs are hydrogen. In this regard, the dibenzo-p-dioxin derivative may comprise the compound of Chemical Formula 3 in an amount of 10˜60 wt. %, the compound of Chemical Formula 5 in an amount of 5˜30 wt. %, the compound of Chemical Formula 6 in an amount of 1˜40 wt. %, the compound of Chemical Formula 8 in 1˜40 wt. %, and the compound of Chemical Formula 10 in an amount of 10˜40 wt. %.

In yet another further preferred embodiment, the dibenzo-p-dioxin derivative of the present invention may comprise two or more compounds selected from the group consisting of compounds represented by Chemical Formulas 1 to 10, with the proviso that Rs are hydrogen. In this regard, the dibenzo-p-dioxin derivative may comprise the compound of Chemical Formula 1 in an amount of 0.1-6 wt %, the compound of Chemical Formula 2 in an amount of 0.1-6 wt %, the compound of Chemical Formula 3 in an amount of 1-30 wt %, the compound of Chemical Formula 4 in an amount of 0.5-20 wt %, the compound of Chemical Formula 5 in an amount of 20-40 wt %, the compound of Chemical Formula 6 in an amount of 1-30 wt %, the compound of Chemical Formula 7 in an amount of 0.5-20 wt %, the compound of Chemical Formula 8 in an amount of 1-30 wt %, the compound of Chemical Formula 9 in an amount of 0.1-10 wt %, or the compound of Chemical Formula 10 in an amount of 0.1-12 wt %, based on the total weight of the composition.

In a yet still preferred embodiment, the dibenzo-p-dioxin derivative of the present invention may comprise two or more compounds selected from the group consisting of compounds represented by Chemical Formulas 1, 2, 3, and 10, with the proviso that Rs are hydrogen. In this regard, the dibenzo-p-dioxin derivative may comprise the compound of Chemical Formula 1 in an amount of 5˜30 wt %, the compound of Chemical Formula 2 in an amount of 1˜30 wt %, the compound of Chemical Formula 3 in an amount of 50˜70 wt %, and the compound of Chemical Formula 10 in an amount of 5˜30 wt %.

The composition or the method of the present invention may be applied to an anticancer therapy/therapies for a cancer selected from the group consisting of, but not limited to, breast cancer, ovarian cancer, lung cancer, liver cancer, stomach cancer, prostate cancer, thyroid cancer, colorectal cancer, uterine cervical cancer, and a combination thereof.

The anticancer therapy to which the composition or the method of the present invention may be applied may be chemotherapy. This chemotherapy may be carried out with an anticancer agent selected from the group consisting of, but not limited to, doxorubicin, cisplatin, paclitaxel, and a combination thereof. Alternatively, the anticancer therapy in combination with which the composition or the method of the present invention can be used may be radiotherapy.

By augmenting the apoptotic effect of anticancer therapy, suppressing the cardiomyopathy caused by anticancer therapy, alleviating the anorexia caused by anticancer therapy, and/or protecting the kidney or liver from renal toxicity or hepatotoxicity induced by anticancer therapy, the composition or the method of the present invention maximizes therapeutic effects of the anticancer therapy.

The term “animal,” as used herein, includes a mammalian or a non-mammalian. Examples of suitable mammalian may include, without limit, human, rodents, companion animals, livestock, and primates. Suitable rodents 10 may include, but are not limited to, mice, rats, hamsters, gerbils, and guinea pigs. Suitable companion animals may include, but are not limited to, cats, dogs, rabbits, and ferrets. Suitable livestock may include, but are not limited to, horses, goats, sheep, swine, cattle, llamas, and alpacas. Suitable primates may include, but are not limited to, chimpanzees, lemurs, macaques, marmosets, spider monkeys, squirrel monkeys, and 15 vervet monkeys. Examples of suitable non-mammalian subject may include, without limit, birds, reptiles, amphibians, and fish. Non-limiting examples of birds include chickens, turkeys, ducks and geese.

The daily dose of the composition or the dibenzo-p-dioxin derivative according to the present invention is preferably on the order of 10˜5000 mg. However, the daily dose is not limited to this range, and may vary depending on various factors including the patient's weight, age, and gender, the state of health, diet, the time of administration, the route of administration, excretion rate, and the severity of a disease.

So long as it may bring the composition of the present invention into a target tissue, any administration route may be taken. For example, the composition of the present invention may be administered orally, intraperitoneally, intravenously, intramuscularly, subcutaneously, intradermally, intranasally, intrapulmonary, intrarectally, intravesicularly, or intradurally.

The composition of the present invention may be administered once a day or two or more times a day at regular intervals of time, and may be used in combination with an anticancer therapy.

The compounds used as active ingredients of the composition or the method for improving anticancer therapy may be obtained using a typical method, for example, may be synthesized from commercially available reagents or may be isolated from natural products, particularly from marine plants.

A better understanding of the present invention may be obtained through the following examples which are set forth to illustrate, but are not to be construed as limiting, the present invention.

### Example 1

### Isolation and Purification of Compounds 1 to 10 from Brown Algae

1-1: Extraction of Polyphenols from Brown Algae

After fresh Eisenia bicyclis (1 kg) was extruded, the extruder was stirred with 95% ethyl alcohol (4 L) at room temperature for 30 min. The mixture was filtered, and the filtrate was dried to produce 58 g of a brown powder. After the dried brown powder was dissolved in 50 weights of distilled water (50° C.), PVPP (polyvinyl pyrrolidone) resin (10 times as large as the weight of the dried powder) was added to the solution and stirred at 50° C. for 1 hr. The PVPP resin was filtered and washed with a sufficient amount of distilled water (5 times as large as the weight of the PVPP resin). The washed PVPP resin was added with 95% ethanol (3 times as large as the weight of the PVPP), stirred at room temperature for 30 min, and filtered. The liquid filtrate was dried to afford 8 g of blackish brown powder. Total polyphenol contents were 96.7% which was determined by a Follin's reagent.

1-2: Isolation of Compounds 1 to 10 from the Purified Polyphenols

The polyphenols obtained in Example 1-1 was filtered through a 0.2 μm membrane filter before being loaded to an HP ODS Hypersil column for high performance liquid chromatography eluting with a linear gradient from 15% to 70% ethanol in distilled water at a flow rate of 1.0 ml/min to isolate 10 active fractions. They were identified as the following compounds 1 to 10:

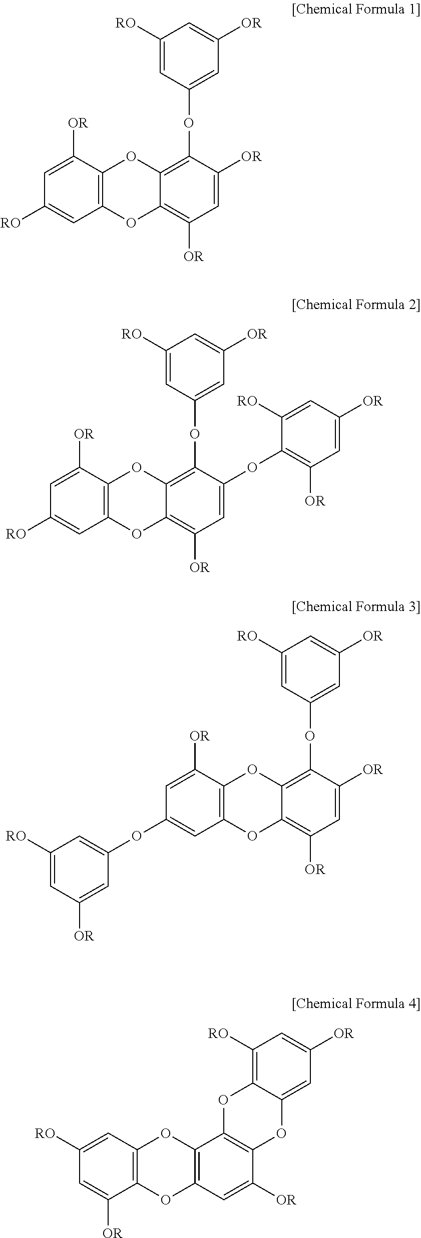

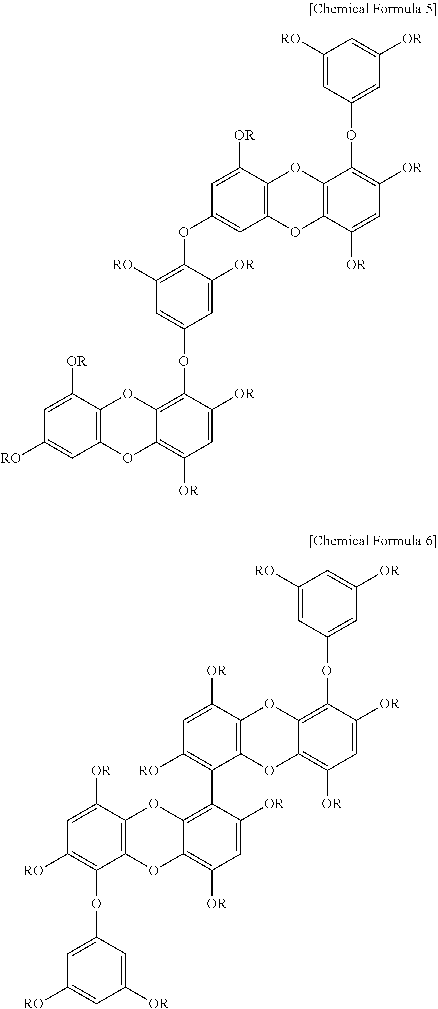

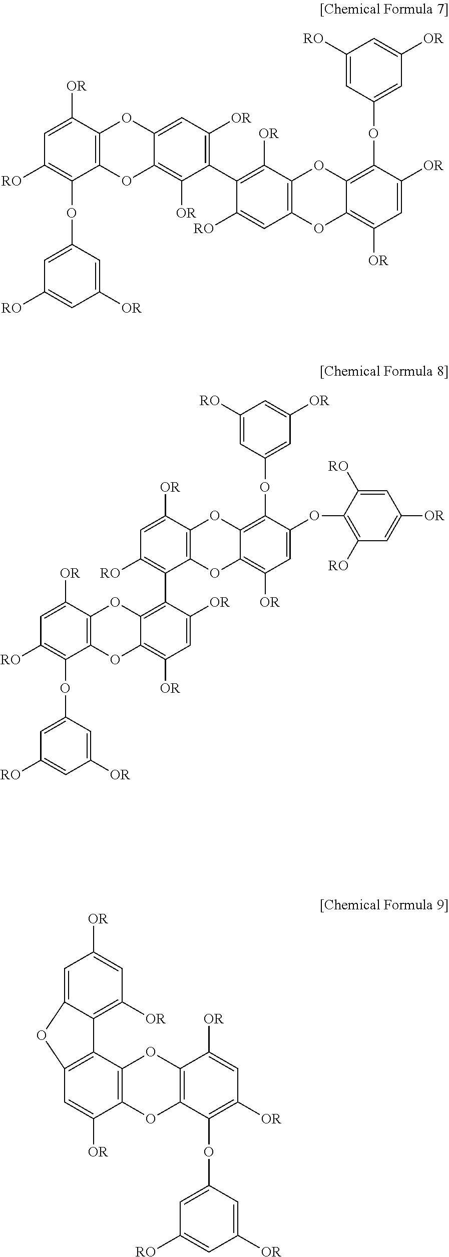

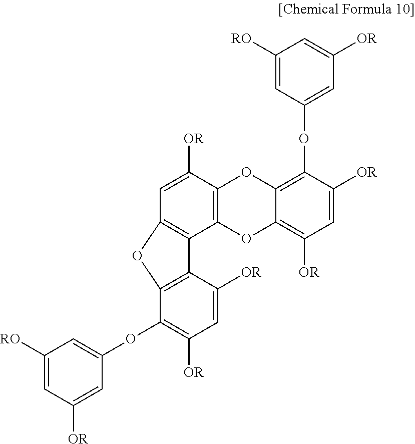

wherein R is hydrogen.

### Example 2

### Preparation of Compositions for Enhancing Therapeutic Effect of Anticancer Therapies, Comprising Combinations of Compounds 1 to 10

Compositions 1 to 5 for enhancing therapeutic effect of anticancer therapies were prepared as shown in Table 1, below.

TABLE 1

| Composition | Content (In Cpds. 1to 10, R = H, weight ratio) |
|---|---|
| Composition 1 | Cpd. 3:Cpd. 5:Cpd. 6:Cpd. 8 = 25:35:20:20 |
| Composition 2 | Cpd. 5:Cpd. 6:Cpd. 7:Cpd. 8 = 43:22:12:23 |
| Composition 3 | Cpd. 3:Cpd. 5:Cpd. 6:Cpd. 8:Cpd. 10 = |
| | 50:15:10:10:15 |
| Composition 4 | Cpd. 1:Cpd. 2:Cpd. 3:Cpd. 4:Cpd. 5:Cpd. 6:Cpd. |
| | 7:Cpd. 8:Cpd. 9:Cpd. 10 = 5:5:20:3:25:15:8:12:2:5 |
| Composition 5 | Cpd. 1:Cpd. 2:Cpd. 3:Cpd. 10 = 16:8:60:16 |

### Reference Example 1

### Inhibitory Effect of the Compounds and the Compositions on Proliferation of Cancer Cell

To examine the inhibitory effect thereof on proliferation of cancer cells, compounds 1 to 10 and compositions 1 to 5 were compared with cisplatin, known as an anticancer agent for ovarian cancer.

Ovarian endothelia carcinoma cell lines (SKOV3, A2780) were incubated in minimum essential medium [MEM] supplemented with 10% FBS, 100 units/ml antibiotic antimycotic, and 2.2 g/L sodium bicarbonate at 37° C. for 24 hrs in 96-well plates. The ovarian carcinoma cell lines were seeded at a density of 1×104 cells/well into 96-well plates and allowed to adhere to the bottom during incubation for 24 hrs. Each of Cisplatin, compounds 1-10, and compositions 1-5 in dimethyl sulfoxide (DMSO) was added in a concentration of 80 μg/ml (A2780) or 120 μg/ml (SKOV3). In this regard, DMSO had a final concentration of 0.1% which was too low to affect growth and death of the cell lines. Following incubation with cisplatin, the compounds, and the compositions for 24 hrs, their inhibitory activity against cancer cells was measured using an MTT assay. The cells in each well were treated with 5 mL of an MTT reagent (1 mg/ml) for 4 hrs in a dark condition. After the medium was aspirated, the MTT fromazan thus formed was dissolved in 100 mL of DMSO, and absorbance was measured at 540 nm using spectrophotometer. The half maximal inhibitory concentration (IC50) of the compounds, the compositions and cisplatin were calculated and summarized in Table 2, below.

TABLE 2

IC50

| Sample | SKOV3 | A2780 |
|---|---|---|
| Cisplatin | 12.30 | 6.01 |
| Cpd. 1 | 120.40 | 80.05 |
| Cpd. 2 | 115.04 | 76.04 |
| Cpd. 3 | 116.08 | 77.06 |
| Cpd. 4 | 115.03 | 81.02 |
| Cpd. 5 | 114.03 | 79.04 |
| Cpd. 6 | 124.02 | 77.40 |
| Cpd. 7 | 120.01 | 76.34 |
| Cpd. 8 | 119.04 | 75.03 |
| Cpd. 9 | 110.90 | 72.09 |
| Cpd. 10 | 112.08 | 73.98 |
| Composition 1 | 120.05 | 80.03 |
| Composition 2 | 124.05 | 81.06 |
| Composition 3 | 125.03 | 82.04 |
| Composition 4 | 118.04 | 79.09 |
| Composition 5 | 116.07 | 79.99 |

As shown in Table 2, the compounds and the compositions showed higher IC50 values, compared to the anticancer agent Cisplatin. That is, the compounds or the compositions can only inhibit the cell proliferation effectively at the high concentrations (80-120 μg/ml). Hence, this result suggests that combination of low concentration of the compounds and compositions with Cisplatin, (cis-Diammineplatinum (II) dichloride; Sigma-Aldrich, Co, LLC, St. Louis, Mo., USA) would be better for enhancing the efficacy of inhibition on cell proliferation.

### Experimental Example 1

### Effects on Apoptosis by Co-Treatment of Inventive Compositions with an Anti Cancer Agent, Cisplatin

The effects of the composition of the present invention on cell cycle and cell death (apoptosis) were investigated by co-treatment of the composition 5 prepared in Example 2 with an anticancer agent for ovarian cancer., Cisplatin.

Apoptotic effects were assessed using a PI staining method. Propidium iodide (PI) is a fluorescent molecule that can bind to DNA of dead cells to evaluate cell cycle and cell viability.

Ovarian carcinoma cells (SKOV3) were incubated at 37° C. for 24 hrs in minimum essential medium [MEM] supplemented with 10% FBS, 100 units/ml antibiotic antimycotic, and 2.2 g/L sodium bicarbonate in 96-well plates. The ovarian carcinoma cell line was seeded at a density of 1×104 cells/well into 96-well plates and allowed to adhere to the bottom during incubation for 24 hrs. Then, the cells were treated with Cisplatin (3 μM or 5 μM), the composition 5 (50 μg/ml, or 100 μg/ml), or Cisplatin+the composition (low dose and high dose) for 48 hrs. Afterwards, the cells were washed with PBS, and detached with trypsin-EDTA, followed by centrifugation at 1,000 rpm for 10 min. The cells were fixed with 70% ethanol at 4° C. for 24 hrs. After removal of the ethanol, the cells were washed many times with PBS, suspended in PBS containing RNaseA, and reacted with a PI reagent for 10 min at room temperature in a dark condition. Dead cells were counted using Fluorescence Activated Cell Sorter (FACS), and the results are summarized in Table 3, below.

TABLE 3

| | Treated with | Apoptosis (%) |
|---|---|---|
| Individual | Cisplatin, 3 μM | 3.0 |
| | Cisplatin, 5 μM | 3.5 |
| | Composition 5, 50 μg/ml | 3.3 |
| | Composition 5, 100 μg/ml | 23.4 |
| Combined | Cisplatin, 3 μM + Composition 5, | 16.2 |
| | 50 μg/ml | |
| | Cisplatin, 5 μM + Composition 5, | 21.8 |
| | 50 μg/ml | |
| | Cisplatin, 3 μM + Composition 5, | 37.8 |
| | 100 μg/ml | |
| | Cisplatin, 5 μM + Composition 5, | 51.2 |
| | 100 μg/ml | |

As shown in Table 3, apoptosis remarkably increased when Cisplatin was co-treated with the composition 5. In addition, the apoptosis increased at the high concentration of Cisplatin and the composition 5. Therefore, it was concluded that co-treatment of the inventive composition with Cisplatin significantly elevated the efficacy of the apoptosis by the synergistic effects of both compounds.

### Experimental Example 2

### Cardioprotective Effect of the Inventive Compositions on Adriamycin Induced Cardiomyopathy

Adriamycin (also called, Doxorubicin) is one of the most popular anticancer agents that have been used since the 1960s for the treatment of a variety of cancers. Despite their efficacy as anticancer agents, there has been limited in use by the development of life-threatening cardiomyopathy. Due to this adverse effect, the anticancer agent can only be used in a limited amount. The composition of the present invention was examined for suppression effects against adriamycin-induced cardiomyopathy.

A total of 25 male mice (33±5 g, four-week-old) were divided into four groups: three test groups of seven, and one control group of four. Adriamycin (tradename: Doxorubicin Hydrochloride; Sigma-Aldrich, Co, LLC; St. Louis, Mo., USA) in distilled water was intraperitoneally injected at a dose of 1.25 mg/kg once a week for six weeks in the adriamycin group. The other two experimental groups, low-dose and high-dose group, were orally administered with composition 4 at a dose of 32 mg/kg and 64 mg/kg, respectively, everyday for one week, and then intraperitoneally injected with adriamycin in distilled water at a dose of 1.25 mg/kg once a week for six weeks. The control group of 4 mice was injected once a week for six weeks with physiological saline. During the experiment, the mice were given access to food and water ad libitum.

The mice were generally anesthetized before and after the six-week experiment, and subjected to two-dimensional echocardiography using a 15-MHz transducer to measure left ventricular end-diastolic dimension (LVEDD) and left ventricular end-systolic dimension (LVESD), left ventricular end-diastolic posterior wall thickness (PW), and left ventricular end-systolic posterior wall thickness (PW). From these measurements, relative wall thickness (RWT), fraction shortening (FS), and left ventricular ejection fraction (LVEF) were calculated. The echocardiographic data are expressed as mean value±SD of five or higher independent measurements. The results are summarized in Table 4, below.

TABLE 4

| | | | | | |
|---|---|---|---|---|---|
| LVEDD (mm) | LVESD (mm) | PW (mm) | RWT (mm) | FS (%) | LVEF (%) |

Adriamycin Group

| | | | | | | |
|---|---|---|---|---|---|---|
| Week 0 (Baseline) | 5.7 ± 0.2 | 2.4 ± 0.3 | 1.6 ± 0.2 | 0.5 ± 0.2 | 59.0v4.7 | 85.5v3.3 |
| Week 6 | 6.8v0.3 | 3.8 ± 0.3 | 1.4 ± 0.3 | 0.4 ± 0.1 | 44.2 ± 3.2 | 70.3 ± 0.3 |
| Δ(6 − 0) | 1.1 ± 0.1 | 1.4 ± 0.0 | 0.2 ± 0.1 | 0.1 ± 0.1 | 14.4 ± 1.5 | 15.2 ± 3.0 |

Low-dose Group (Adriamycin + Composition 4, 3.2 mg/kg)

| | | | | | | |
|---|---|---|---|---|---|---|
| Week 0 (Baseline) | 5.8 ± 0.2 | 2.1 ± 0.2 | 1.6 ± 0.1 | 0.6 ± 0.0 | 63.6 ± 3.1 | 88.7 ± 1.8 |
| (Week 6 | 7.1 ± 0.7 | 3.4 ± 0.6 | 1.6 ± 0.1 | 0.4 ± 0.1 | 52.7 ± 6.3 | 78.8 ± 5.8 |
| Δ(6 − 0) | 1.3 ± 0.5 | 1.3 ± 0.4 | 0.0 ± 0.0 | 0.2 ± 0.2 | 10.7 ± 3.2 | 9.9 ± 1.5 |

High-dose Group (Adriamycin + Composition 4, 6.4 mg/kg)

| | | | | | | |
|---|---|---|---|---|---|---|
| Week 0 (Baseline) | 5.8 ± 0.5 | 2.4 ± 0.8 | 1.5 ± 0.1 | 0.5 ± 0.1 | 58.7 ± 10.2 | 84.8 ± 7.3 |
| Week 6 | 6.3 ± 0.4 | 2.8 ± 0.5 | 1.5 ± 0.1 | 0.5 ± 0.1 | 56.4 ± 5.2 | 82.6 ± 5.2 |
| Δ(6 − 0) | 0.5 ± 0.1 | 0.4 ± 0.3 | 0.0 ± 0.0 | 0.0 ± 0.0 | 2.3 ± 5.0 | 2.2 ± 2.1 |

As shown in Table 4, the adriamycin group showed cardiomyopathy with significant decrease in left ventricular posterior wall thickness (PW) and in fractional shortening (FS) after 6 weeks of study. However, low-dose group showed the improvement in cardiomyopathy and there were no significant changes on heart either functionally or morphologically in high-dose group after 6 weeks. Accordingly, it is concluded that the composition of the present invention has a potent cardioprotective effect against adriamycin-induced cardiomyopathy.

### Experimental Example 3

### Effect on Tumor Suppression and Anorexia by Co-Treatment of the Inventive Composition with Cisplatin in Ovarian Carcinogenesis

Ovarian cancer is the fifth most common cancer among women in the world, and is the deadliest of gynecologic cancers. Over 200,000 new ovarian cancers are diagnosed in worldwide yearly. The incidence of ovarian cancer has increased rapidly in Korea. In spite of extensive researches, there have been no significant achievements in the prevention and treatment of ovarian cancer. Signs and symptoms of ovarian cancer are frequently absent at early stages and when they manifest they may be subtle. Most cases of ovarian cancer are not diagnosed until they reach advanced stages because it lacks any clear early detection or screening test, with a five-year rate of as low as around 15% after diagnosis. Treatment usually involves surgery, and chemotherapy with the platinum/taxin anticancer agents. In primary chemotherapy, ovarian cancer exhibits a chemical sensitivity of about 80%, but in most cases, recurs because of drug resistance, which accounts for such a low survival rate. Hence, there is a pressing need for a cancer sensitizer that can alleviate resistance to an anticancer agent administered together therewith, with the suppression of side effects of the anticancer agent. In this context, the composition, prepared in Example 2, for improving anticancer therapy in accordance with the present invention was examined for tumor suppression and anorexia improvement when administered, together with Cisplatin, for ovarian cancer.

The ovarian carcinoma cell line SKOV3 was detached from a culture dish using trypsin-EDTA, and diluted to a density of 1×10 cells/150 in an RPMI medium supplemented with 10% FBS, followed by subcutaneous injection into the right flank of Balb/c nu/nu mice using a syringe. When the tumors thus formed and reached a size of 100˜150 mm³, as measured by a caliper, the mice were divided into 5 groups according to the tumor size: control cisplatin, composition, low-dose and high-dose group. The cisplain and composition group were administrated with 3 mg/kg of cisplatin and 150 mg/kg of the composition 3, respectively, for 4 weeks. The low-dose and high-dose group was treated with composition 3 (75 mg/kg for low-dose, 150 mg/kg for high-dose) and Cisplatin (3 mg/kg), for 4 weeks. Composition 3 was orally administered while Cisplatin was intraperitoneally injected. The control group was treated with PBS. During the experimental period, the tumor size, body weight and dietary intake of mice in each group was recorded three times per week. Results are shown in Table 5 and Table 6.

After 4 weeks, the animals were sacrificed and blood was taken from the animals. Tumors were e.xcised from the animals and measured for size and weight. The results are given in Table 5.

TABLE 5

| Diet Intake (gram) | Week 0 | Week 1 | Week 2 | Week 3 | Week 4 |
|---|---|---|---|---|---|
| Control group | 5.0 | 5.3 | 5.1 | 4.9 | 4.9 |
| Cisplatin group | 4.8 | 4.6 | 4.0 | 3.7 | 4.1 |
| Composition group | 4.2 | 4.8 | 5.1 | 5.8 | 5.6 |
| low-dose group | 4.7 | 5.0 | 5.2 | 5.1 | 4.9 |
| high-dose group | 4.3 | 5.1 | 5.3 | 5.2 | 5.1 |

TABLE 6

| | Tumor Volume (mm3) | | | | Tumor Weight |
|---|---|---|---|---|---|
| | Week | Week | Week | Week | after 4 Weeks |
| | 1 | 2 | 3 | 4 | (gram) |
| Control | 100 | 140 | 160 | 180 | 2.2 |
| Cisplatin alone | 100 | 134 | 144 | 162 | 1.7 |
| Composition 3 | 100 | 126 | 142 | 160 | 1.3 |
| Cisplatin + | 100 | 124 | 140 | 150 | 1.1 |
| low-dose composition | | | | | |
| Cisplatin + | 100 | 120 | 126 | 124 | 0.8 |
| high-dose composition | | | | | |

As shown in Table 5, the cisplatin group showed the decrease in dietary intake during the experimental periods compared to control group, whereas the composition group showed increase in dietary intake compared to the control group. No reduction in dietary intake was detected in the low-dose and high-dose group. Thus, the composition of the present invention is shown to reduce the anorexia caused by Cisplatin when administered together.

As shown in Table 6, the tumor size in cisplatin group was slightly reduced compared to the control group. However, the tumor size in the composition group was significantly reduced compared to the control. In addition, the composition reduces the tumor size in a dose-dependent manner. Therefore, a combination of Cisplatin and the composition of the present invention was observed to suppress the growth of ovarian carcinoma tumors more effectively, compared to Cisplatin or the composition alone, as measured for tumor weight after administration for 4 weeks, indicating that the composition of the present invention can be useful as an agent for augmenting effects of anticancer therapy.

### Experimental Example 4

### Nephroprotective and Hepatoprotective Activity of the Inventive Composition Against Anticancer Agent

Serum AST (GOT) and ALT (GPT) are commonly measured clinically as a part of a diagnostic evaluation of hepatocellular injury, to determine liver health. These enzymes are associated with liver parenchymal cells, and released into the blood when the cell membranes of hepatocytes are damaged. Thus, blood concentrations and relative ratios of these enzymes can be used to determine hepatotoxicity. AST (GOT) and ALT (GPT) in the blood samples taken in Experimental Example 3 were measured, and the results are given in Table 7.

After entering the body, various chemicals are excreted through the kidneys. Thus, after various chemicals are metabolized by enzymes, the resulting wastes are removed by the kidneys. Serum creatinine and BUN are important indicators of renal health. Measurements of creatinine and BUN levels are given in Table 7, below.

TABLE 7

| | AST | ALT | Creatine | BUN |
|---|---|---|---|---|
| | (U/L) | (U/L) | (mg/L) | (mg/L) |
| Control | 86 | 40 | 0.6 | 24 |
| Cisplatin alone | 92 | 42 | 1.2 | 37 |
| Composition 3 alone | 86 | 41 | 0.4 | 26 |
| Cisplatin + low-dose composition | 88 | 43 | 0.9 | 31 |
| Cisplatin + high-doe composition | 90 | 45 | 0.7 | 29 |

As seen in Table 7, AST (GOT) and ALT (GPT) were not significantly changed by administration of Cisplatin or composition group, compared to the control group. The combinations of Cisplatin and the composition did not cause a significant change in AST (GOT) and ALT (GPT), either. Thus, the compositions of the present invention are free of hepatotoxicity.

On the other hand, Cisplatin was observed to induce nephrotoxicity in view of creatinine and BUN. The composition, even when administered in combination with Cisplatin, did not evoke a significant change in creatinine and BUN, compared to the control. Therefore, the composition of the present invention is observed to protect the kidneys from the toxicity of Cisplatin.

Although the embodiments of the present invention have been disclosed for illustrative purposes, it will be appreciated that the present invention is not limited thereto, and those skilled in the art will appreciate that various modifications, additions and substitutions are possible, without departing from the scope and spirit of the invention. Accordingly, any and all modifications, variations or equivalent arrangements should be considered to be within the scope of the invention, and the detailed scope of the invention will be disclosed by the accompanying claims.

What is claimed is:

1. A method for enhancing therapeutic effect of anticancer therapies for an animal, comprising administering one or more dibenzo-p-dioxin derivative in an effective amount to the animal in anticancer therapies,
wherein said dibenzo-p-dioxin derivative is one or more selected from the group consisting of compounds represented by the following Chemical Formulas 1 to 10: 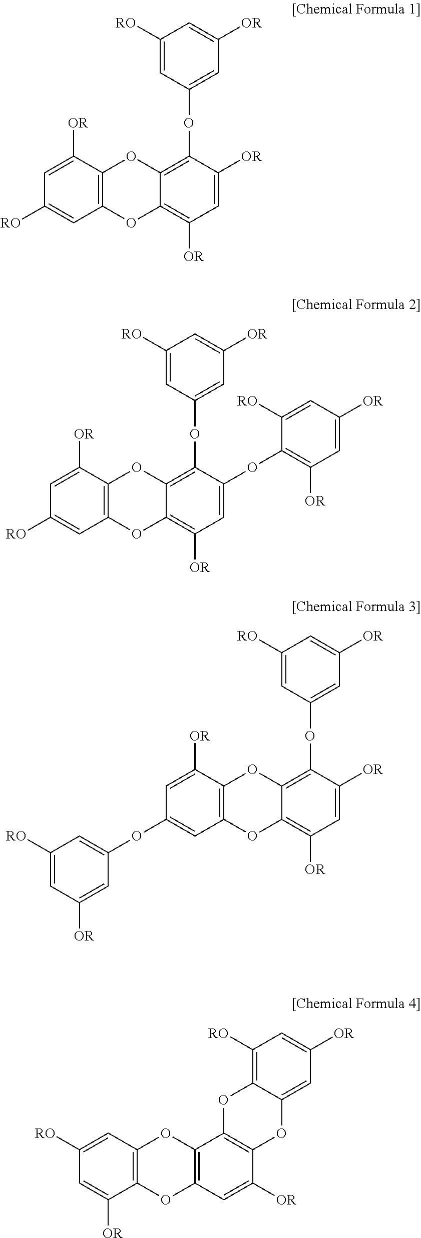 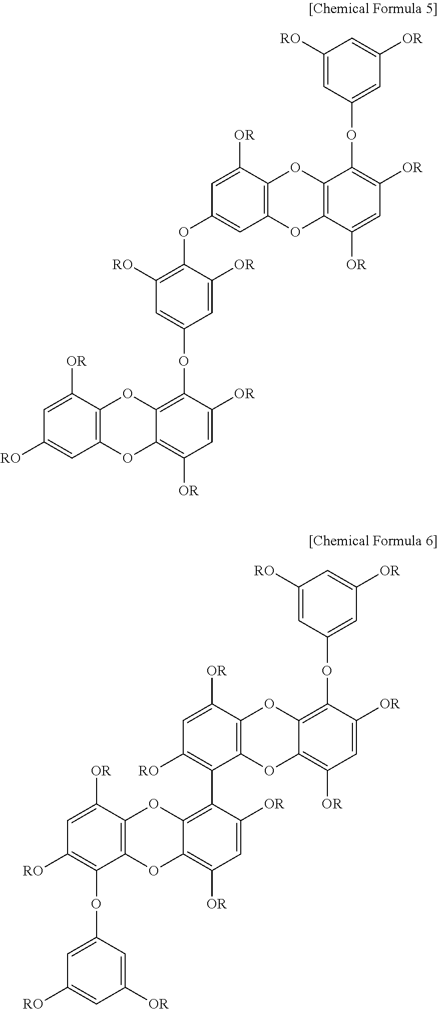 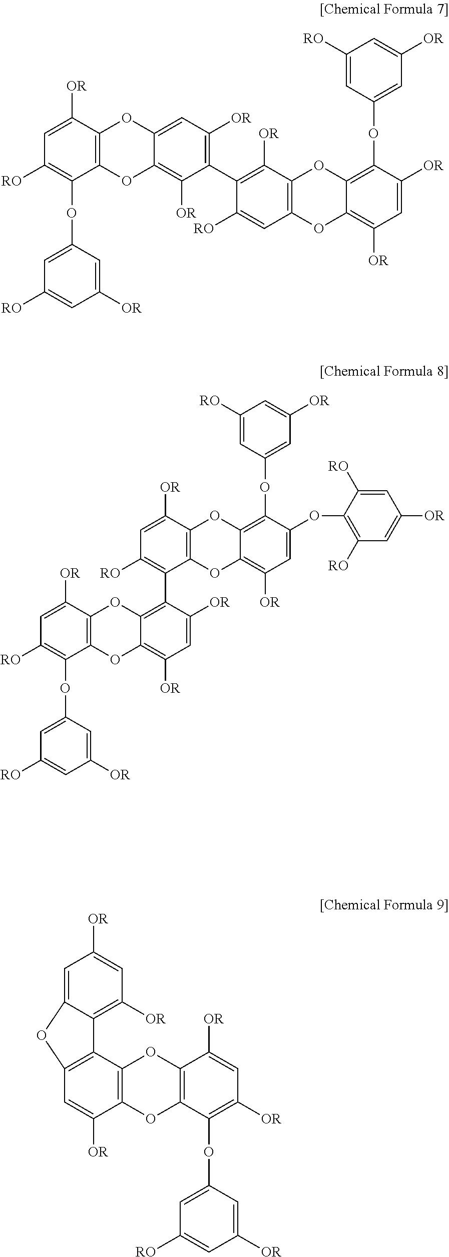 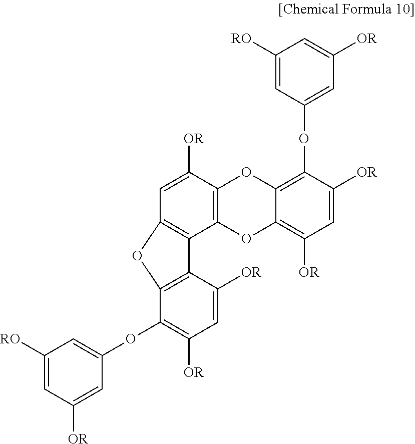
wherein, Rs are independently hydrogen, alkyl of C1˜C5, alkenyl of C2˜C5, phenyl, phenylalkyl of C7˜C12, alkanoyl of C2˜C20, alkenoyl of C3˜C20, hydroxyphenyl, dihydroxyphenyl or trihydroxyphenyl.

2. The method of claim 1, wherein Rs are hydrogen.

3. The method of claim 1, wherein the dibenzo-p-dioxin derivative comprises:
compounds respectively represented by Chemical Formulas 3, 5, 6, and 8;
compounds respectively represented by Chemical Formulas 5, 6, 7, and 8;
compounds respectively represented by Chemical Formulas 3, 5, 6, 8, and 10;
compounds respectively represented by Chemical Formulas 1, 2, 3, 4, 5, 6, 7, 8, 9, and 10; or
compounds respectively represented by Chemical Formulas 1, 2, 3, and 10.

4. The method of claim 1, being applied to the anticancer therapies for breast cancer, ovarian cancer, lung cancer, liver cancer, stomach cancer, prostate cancer, thyroid cancer, colorectal cancer, or uterine cervical cancer.

5. The method of claim 1, wherein the anticancer therapies are chemo therapies with an anticancer agent selected from the group consisting of doxorubicin, cisplatin, paclitaxel, and a combination thereof.

6. The method of claim 1, wherein the anticancer therapy is a radiotherapy.

7. The method of claim 1, wherein the dibenzo-p-dioxin derivative functions to augment an apoptotic effect of the anticancer therapies on cancer cells.

8. The method of claim 1, wherein the dibenzo-p-dioxin derivative functions to alleviate cardiomyopathy caused by the anticancer therapies.

9. The method of claim 1, wherein the dibenzo-p-dioxin derivative functions to alleviate the anorexia caused by the anticancer therapies.

10. The method of claim 1, wherein the dibenzo-p-dioxin derivative functions to protect a kidney or a liver from nephrotoxicity or hepatotoxicity induced by the anticancer therapies.
